(19) United States
(12) Patent Application Publication
(10) Pub. No.: US 2025/0356967 A1
(43) Pub. Date: Nov. 20, 2025

(54) ELECTRONIC HEALTH RECORD SYSTEM

(71) Applicant: IRYOU JYOUHOU GIJYUTU KENKYUSHO CORPORATION, Fukuoka (JP)

(72) Inventor: Shinkichi HIMENO, Fukuoka (JP)

(73) Assignee: IRYOU JYOUHOU GIJYUTU KENKYUSHO CORPORATION, Fukuoka (JP)

(21) Appl. No.: 18/867,324

(22) PCT Filed: Aug. 12, 2023
(86) PCT No.: PCT/JP2023/029385

§ 371 Date: Nov. 19, 2024

(30) Foreign Application Priority Data
Aug. 18, 2022 (JP) 2022130329

(51) Int. Cl. G16H 10/60

(52) U.S. Cl. CPC G16H 10/60

(57) ABSTRACT

Data interoperability is to be ensured when support modules in accordance with work procedures of each medical or nursing care institution and each occupation is developed, and electronic health record elements developed/corrected in a hospital are registered in an electronic health record element database, thereby enabling the electronic health record elements to be shared with other medical/nursing care institutions while backing up the electronic health record elements in the hospital. In an electronic health record system including a combination of support software (support modules) by occupation that supports creation of documents for each of a plurality of occupations in medical/nursing care, the electronic health record system enables documents created by the support modules to be referenced between any combination of the support modules.

### CROSS-REFERENCE TO RELATED APPLICATIONS

The present application is a National Stage Application of PCT International Application No.: PCT/JP2023/029385 filed on Aug. 12, 2023, which claims priority to Japanese Patent Application 2022-130329, filed in the Japanese Patent Office on Aug. 18, 2022, the disclosure of which is incorporated herein by reference in its entirety.

### TECHNICAL FIELD

The present invention relates to an electronic health record system used in medical and nursing institutions for planning, treatment orders, and recording.

### BACKGROUND ART

In medical and nursing care fields, recording of information obtained using electronic health records, planning based on these, individual test and treatment orders based on the plans, recording of the execution of orders, and the like are systematically performed in order to implement efficient and less erroneous medical and nursing care practices.

These medical and nursing care practices are diverse, and there are methods of recording, planning, and ordering according to each of them.

Methods of handling information, planning, ordering, and recording differ between occupations such as doctors, nurses, care workers, and pharmacists.

In addition, information, plans, orders, and records required for inpatient, outpatient and home healthcare/nursing care are different.

Furthermore, required information, plans, orders, and records are different between hospitals and medical specialties.

Prior art documents related to this application are as follows.

### CITATION LIST

### Patent Literature

Patent Literature 1: JP 2018-151988 A

Patent Literature 2: JP 2018-180731 A

Patent Literature 3: JP 2013-65216 A

Patent Literature 4: JP 6488778 B2

### SUMMARY OF INVENTION

Initially, electronic health records were developed individually for each medical or nursing care institution, with a development company conducting interviews about their working methods and then determining the design specifications accordingly. The development and implementation required six months to a year and a huge cost.

As common specifications were gradually fixed, basic specifications were defined, and development shifted to adding functions requested by individual medical/nursing care institutions.

Although development times and costs have been reduced compared to the past, there are still significant costs associated with duplicate development, and it is difficult to say that the demands of individual medical/nursing care institutions are being fully met due to these cost constraints.

There are systems in which multiple types of support software modularized for each occupation, such as a doctor support module and a nursing support module, are prepared, appropriate modules are selected according to the work in a medical/nursing care institution to configure an electronic health record with a combination of them. Respective modules are often provided by multiple different companies.

However, there is a problem that the content of documents created by different support modules cannot be referenced due to the lack of interoperability between the support modules. For this reason, when a doctor wishes to refer to a nurse's record and reflect it in a doctor article, the doctor needs to first close a doctor support module and start a nursing support module to refer to a nursing record. The doctor needs to write down the nursing record that has been referred to, close the nursing support module, start the doctor support module again, and input the note wrote down into the doctor support module, which is a complicated and inefficient process.

In fact, a pharmacist at a medical institution has three terminals running a doctor support module, a nursing support module, and a pharmacist support module in parallel to improve work efficiency, and works while referring to the information needed.

The present invention has been made to solve such a conventional problem, and a purpose of the present invention is to ensure data interoperability when support modules in accordance with work procedures of each medical or nursing care institution and each occupation is developed, and further to eliminate duplicate development of software by registering a large number of support modules (an electronic health record element database) that ensure data interoperability and by enabling introduction of an electronic health record suitable for the medical or nursing care institution with minimum work, such as combining appropriate support modules and adjusting parameters, to enable introduction of the electronic health record in a short period and at a low cost. Furthermore, by registering electronic health record elements developed/corrected in a hospital in the electronic health record element database, the electronic health record elements are to be enabled to be shared with other medical/nursing care institutions while backing up the electronic health record elements in the hospital.

### Solution to Problem

As a means for achieving the above purposes, an electronic health record system according to claim 1 including a combination of support software (support modules) by occupation that supports creation of documents for each of a plurality of occupations in medical and nursing care, the electronic health record system enabling documents created by the support modules to be referenced between any combination of the support modules, the electronic health record system includes an electronic health record element database that records any number of respective definitions of the support modules, the documents created by the support modules, data relating to sections being input areas used in the documents, and description items used in the documents, which are elements of the electronic health record system, and a means for downloading elements of an electronic health record system necessary for a medical/nursing care facility and for configuring an electronic health record of the medical/nursing care facility.

The electronic health record system according to claim 2 enables an electronic health record element corrected or newly created in a medical/nursing care facility to be registered in the electronic health record element database in the electronic health record system according to claim 1.

### Advantageous Effects of Invention

In an electronic health record system according to claim 1 including a combination of support software (support modules) by occupation that supports creation of documents for each of a plurality of occupations in medical and nursing care, the electronic health record system enables documents created by the support modules to be referenced between any combination of the support modules.

The electronic health record system according to claim 1 includes an electronic health record element database, and it is thereby possible to record any number of respective definitions of the support modules, the documents created by the support modules, sections being input areas used in the documents, and description items used in the documents, which are elements of the electronic health record system.

In addition, it is possible to download electronic health record elements necessary for a medical/nursing care facility to configure an electronic health record of the medical/nursing care facility.

The electronic health record system according to claim 2 enables an electronic health record element corrected or newly created in a medical/nursing care facility among the electronic health record elements to be registered in the electronic health record element database.

### BRIEF DESCRIPTION OF DRAWINGS

FIG. 1 is a hardware configuration diagram of the present invention.

FIG. 2 shows (a) a conventional software configuration diagram and (b) a software configuration diagram of the present invention.

FIG. 3 is an explanatory diagram for documents of various categories and elements of the documents.

FIG. 4 is an occupation list, a staff list, and a list of access to each document category by occupation.

FIG. 5 is a configuration diagram of an electronic health record element database.

FIG. 6 is an explanatory diagram for downloading elements from an electronic health record element database to configure an electronic health record of a hospital.

### DESCRIPTION OF EMBODIMENTS

An electronic health record system according to the present invention includes a server device, a database, and a terminal.

The server device is a known computer device, and includes an arithmetic device, a main storage device, an auxiliary storage device, an input device, an output device, and a communication device.

The arithmetic device, the main storage device, the auxiliary storage device, the input device, the output device, and the communication device are connected to each other via a bus interface.

The arithmetic device includes a known processor capable of executing a command set.

The main storage device includes a volatile memory, such as a RAM, capable of temporarily storing a command set.

The auxiliary storage device includes a non-volatile data storage capable of recording an OS and a program.

The data storage may be, for example, an HDD or an SSD.

The input device is, for example, a keyboard.

The output device is, for example, a display such as an LCD.

The communication device includes a network interface connectable to a network.

The processor of the server device achieves the functions and effects of the devices of the present application.

The system of the present application includes a means (program) for downloading elements of an electronic health record system necessary for a medical/nursing care facility and for configuring an electronic health record of the medical/nursing care facility.

The database according to the present invention may be configured by the auxiliary storage device of the server device, or may be configured by another auxiliary storage device independent of the server device.

The database stores information handled by the electronic health record system.

The terminal according to the present invention has a hardware configuration of a known computer similarly to the server device.

The server device, the database, and the terminal according to the present invention can communicate via a network.

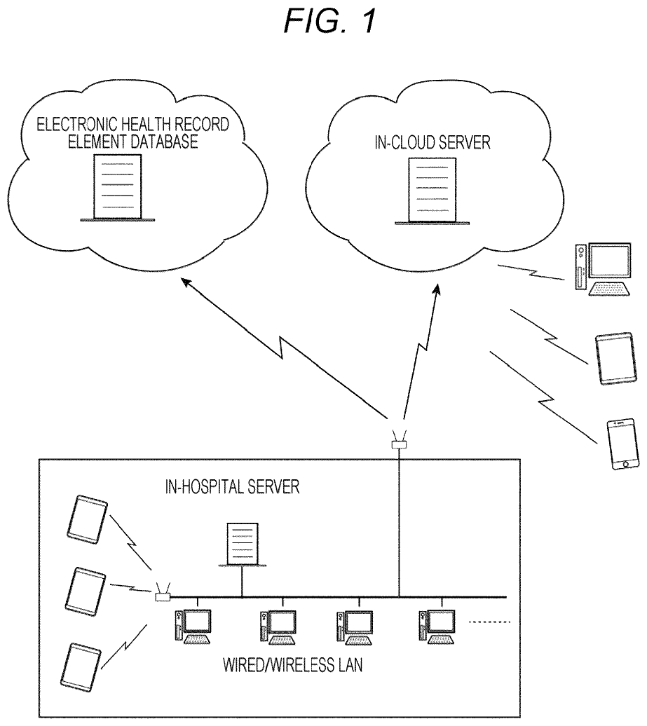

FIG. 1 shows a hardware configuration diagram of the present invention.

An in-hospital server is in a facility and the electronic health record system is operating.

Terminals in the hospital are connected by a wired or wireless LAN.

The terminals are a group of devices such as PCs, tablets, and smartphones.

It is also possible to install a main or backup server on the cloud as necessary.

Devices outside the hospital can also access the in-hospital or in-cloud server via a web line or a public line.

An electronic health record element database, which is also on the cloud, registers/manages elements, such as description items, sections, and document categories constituting an electronic health record, and electronic health record elements necessary for the electronic health record of the hospital are downloaded.

In addition, electronic health record elements created in the hospital are registered and shared with other medical/nursing care institutions. If the electronic health record of the hospital is damaged, the registered electronic health record elements are downloaded to reconfigure the electronic health record.

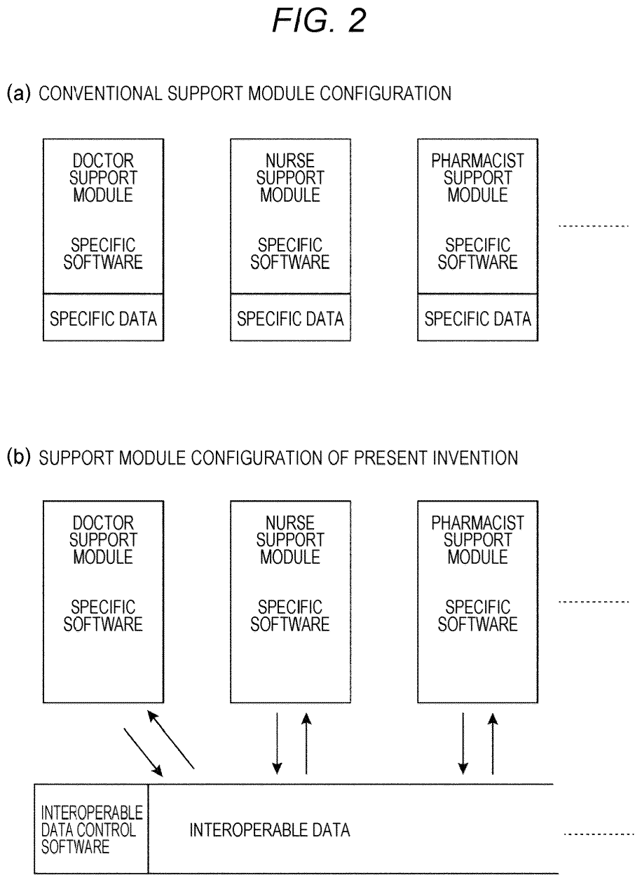

FIG. 2 shows (a) a conventional software configuration diagram and (b) a software configuration diagram of the present invention.
(a) In a conventional support module for an electronic health record, both specific software used in the support module and specific data of the support module created, recorded, and referenced by the specific software were independent from other support modules.

That is, such a conventional support module was only configured by assembling various support modules, like parquetry, and had no interoperability.

For this reason, when a doctor using a doctor support module wished to refer to a nursing record or a comment of a pharmacist, the doctor could not refer directly from the doctor support module, and the doctor needed to first close the doctor support module, start a nurse support module or a pharmacist support module to refer to them, close the support module, and start the doctor support module again, which was a complicated work procedure.
(b) In the present invention, data used in each support module is recorded centrally in a form that can be referenced by other support modules (interoperable data).

The created document data is recorded as interoperable data and is referenced not only from the support module but also from other support modules.

Even if elements, description items, and the like in a document category are different for each document type (document category), as long as the document data is recorded as an object in a format such as XML or JSON, the document data can be managed centrally in a relational database (interoperable data: reiteration).

Interoperable data control software defines a document category and a document internal structure necessary for operation of the interoperable data, and sets an access authority for each occupation and each document category for each occupation.

The interoperable data control software may perform some functions, such as document category definition, within each support module as long as interoperability is ensured.

The development language of each support module is any language as long as interoperability is ensured, such as HTML, BASIC, COBOL, and PYTHON (registered trademark).

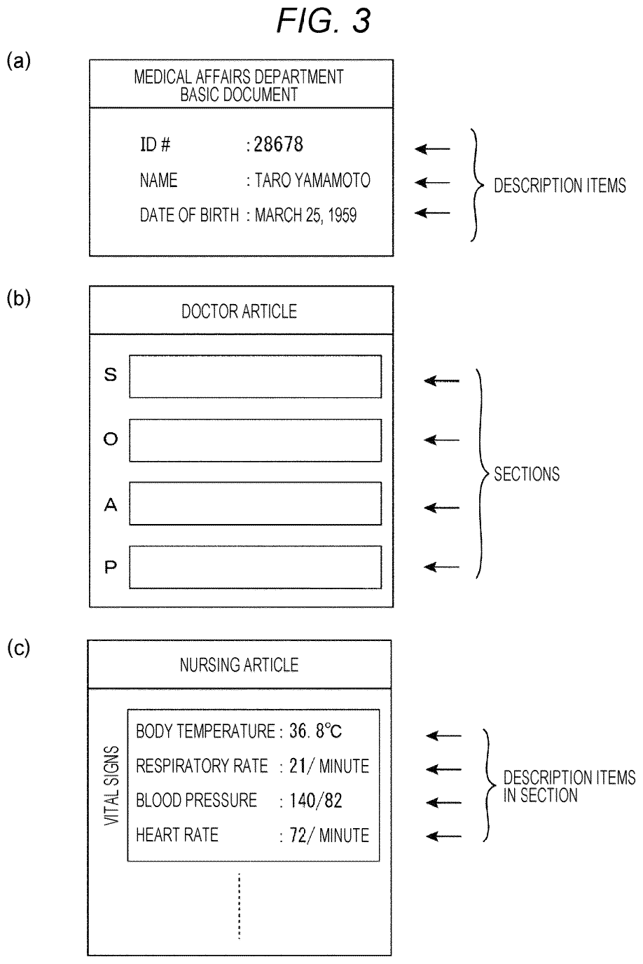

FIG. 3 is an explanatory diagram for documents of various categories and elements of the documents.
(a) This is an example of a medical affairs department basic document. Description items such as a patient ID (ID #), a name, and a date of birth are shown. In this example, the name of a description item and the content are separated by “:”.

This delimiter is a space, “;”,/and the like.
(b) This is an example of a doctor article. There are four free text input areas (sections) of S: Subjective complaints, O: Objective findings, A: Assessment, and P: Plan.
(c) This is an example of a nurse article. In a section called a vital sign, description items of a body temperature, a respiratory rate, a blood pressure, and a heart rate are shown. Each description item may be a label that is a marker for text input, as in the present example. However, if the description item is made into an object and has a filtering function for an input, such as setting a numeric value range and warning of an abnormal value, or defining and selecting a list of options, for more efficient and highly accurate input.

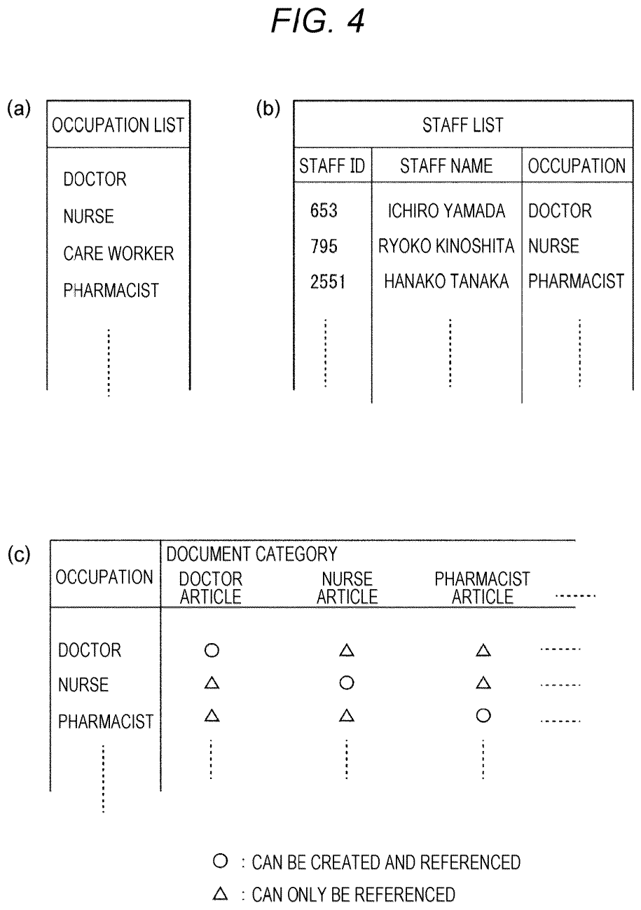

FIG. 4 shows (a) an occupation list, (b) a staff list, and (c) a list of access to each document category by occupation. They are control data managed by the interoperable data control software.

The access authority to a document category varies depending on the occupation of a staff. The allocation of the access authority can be appropriately adjusted by each medical/nursing care institution.

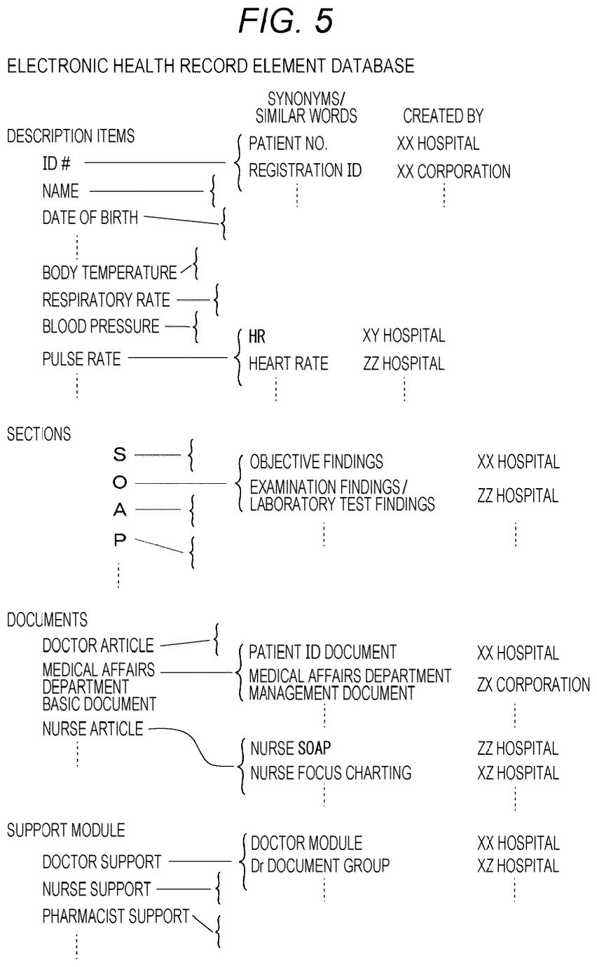

FIG. 5 is a configuration of the content of an electronic health record element database.

A description item group which is a description content of a document, a section group which is an input frame of the document, a document category group, and a support module group are recorded. Synonymous or similar electronic health record elements for any of the description items, sections, document categories, and support modules have been created and registered at various medical/nursing care institutions. Depending on the working environment and target patients of a medical/nursing care institution, there are electronic health record elements suitable for each.

A medical/nursing care institution that wishes to introduce an electronic health record can easily configure the electronic health record of its own hospital by searching the element database shown in FIG. 5, selecting and combining electronic health record elements highly compatible with the hospital, and further setting the access authority shown in FIG. 4.

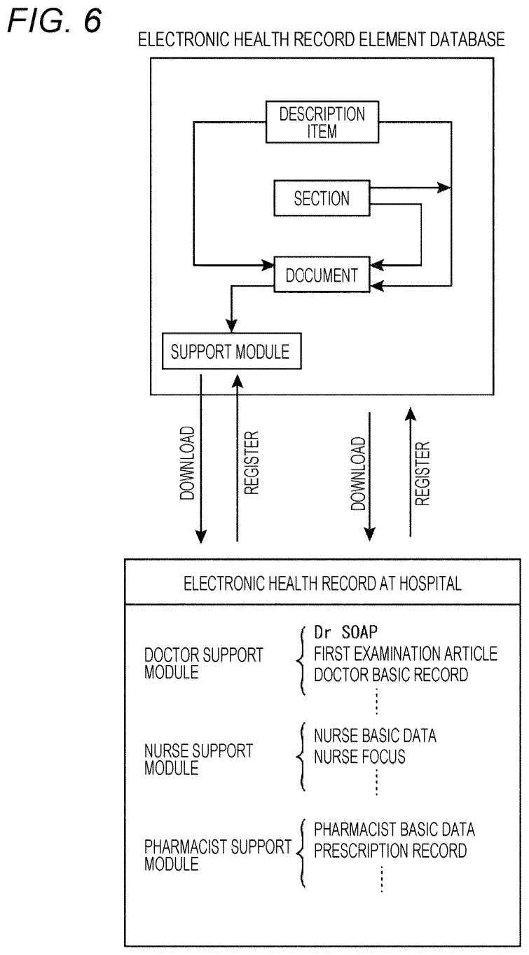

FIG. 6 is an explanatory diagram for downloading electronic health record elements from an electronic health record element database to configure an electronic health record of a hospital.

The document of each category is constituted by description items, sections, and in-section description items. The support module is constituted by a combination of documents of a category created by the occupation.

When an electronic health record is introduced into the hospital, it is easy to download an existing support module from the electronic health record element database, but some of the document categories constituting the support module may be downloaded from a more appropriate electronic health record element database to replace them. In addition, description items and sections of the document category may be similarly downloaded and replaced.

It is needless to say that electronic health record element may be newly created/corrected in the hospital. Furthermore, if a new created/corrected element is registered in the electronic health record element database, which is a backup in the event that the electronic health record of the hospital is damaged, and elements that has been corrected and easy to use can be shared with other medical/nursing care institutions. In order to encourage downloads and registrations, it may be useful to offer an incentive such as money.

It is useful to build the electronic health record element database on a secure cloud such as GitHub (GitHub is a trademark or registered trademark of GitHub Inc.), but other products are applicable as long as they have similar functions and security strength. In addition, an internal server or the like in a medical institution or the like serving as a host may be used.

Although the embodiment has been described above, the specific configuration of the present invention is not limited to the embodiment, and design changes and the like without departing from the gist of the invention are included in the present invention.

For example, in this specification, doctor articles, nurse articles, and pharmacist articles are described, but there are also care workers, laboratory technologists, radiation technologists, rehabilitation therapists, and the like. Basically, it is desirable that documents are managed centrally as interoperable data, but documents that do not need to be shared or are not suitable for sharing may be separately managed by a support module.

1. An electronic health record system including a combination of support software (support modules) by occupation that supports creation of documents for each of a plurality of occupations in medical and nursing care, the electronic health record system enabling documents created by the support modules to be referenced between any combination of the support modules, the electronic health record system comprising:
an electronic health record element database configured to record any number of respective definitions of the support modules, the documents created by the support modules, data relating to sections being input areas used in the documents, and description items used in the documents, which are elements of the electronic health record system; and
a means for downloading elements of an electronic health record system necessary for a medical/nursing care facility and for configuring an electronic health record of the medical/nursing care facility.

2. The electronic health record system according to claim 1, wherein the electronic health record system enables an electronic health record element corrected or newly created in a medical/nursing care facility to be registered in the electronic health record element database.
